(19) United States
(12) Patent Application Publication
(10) Pub. No.: US 2020/0215074 A1
(43) Pub. Date: Jul. 9, 2020

(54) Method of Treatment of Cancer

(71) Applicant: CIPLA LIMITED, Mumbai (IN)

(72) Inventors: Geena MALHOTRA, Mumbai (IN); Kalpana JOSHI, Thane (IN); Jeevan GHOSALKAR, Thane (West) (IN)

(21) Appl. No.: 16/624,439

(22) PCT Filed: Jun. 22, 2018
(86) PCT No.: PCT/IN2018/050409

§ 371 Date: Dec. 19, 2019

(30) Foreign Application Priority Data
Jun. 22, 2017 (IN) 201721021945

(51) Int. Cl. A61K 31/5383; A61K 31/192; A61K 31/35; A61K 31/47; A61K 31/4184; A61K 31/496; A61K 31/4375; A61K 31/506; A61K 31/519; A61K 31/513; A61P 35/04

(52) U.S. Cl. CPC A61K 31/5383; A61K 31/192; A61K 31/35; A61K 31/47; A61K 31/4184; A61K 45/06; A61K 31/4375; A61K 31/506; A61K 31/519; A61K 31/513; A61P 35/04; A61K 31/496

(57) ABSTRACT

Disclosed herein are methods for treating cancer in a subject in need thereof by administering an agent or pharmaceutically acceptable derivative thereof, optionally with another agent, induces prostate apoptosis response-4 (Par-4) production by non-cancerous normal cells, to promote apoptosis in cancer cells.

### CROSS REFERENCE TO RELATED APPLICATIONS

This application is a filing under 35 U.S.C. 371 of International Application No. PCT/IN2018/050409 filed Jun. 22, 2018, entitled “Method of Treatment of Cancer” which claims priority to Indian Patent Application Serial Number 201721021945 filed on Jun. 22, 2017, which applications are incorporated by reference herein in their entirety.

### FIELD OF INVENTION

The present invention relates to methods for treating cancer in a subject by administering a prostate apoptosis response-4 (PAR-4) inducing agent to a subject in need thereof. The PAR-4 inducing agent (generally referred as Secretagogues) is administered in amounts sufficient to induce production and/or secretion of the tumor suppressor PAR-4 by cells, preferably in an amount sufficient to inhibit proliferation and/or metastasis of cancer cells, and/or reduce the recurrence of tumors. The PAR-4 inducing agent may be administered with or without a chemotherapeutic and other anticancer therapy. The PAR-4 inducing agent may also optionally be administered with ionizing radiation.

### BACKGROUND OF INVENTION

The development and progression of cancer is a multistep process involving accumulation of multiple genetic aberrations. Most notable among such aberrations is the loss of apoptotic responses that normally serve as built in checkpoints against the emergence of cell populations with dysfunctional traits or the acquisition of pro-survival mechanisms that override the apoptotic signals. The loss of apoptotic mechanisms often results in abridged response to cancer therapy. As such, alternate or combinatorial approaches that kill cancer cells and induce tumor regression are being actively pursued by researchers and physicians.

Especially difficult to treat are those cancers which are hormonally related and/or are metastatic cancers. These cancers include, e.g., prostate cancer, breast cancer and lung cancer. Melanoma is also difficult to treat and has a low survival rate relative to many other cancers.

An essential feature of anticancer strategies is the selective action against cancer cells, with little or no damage inflicted in normal cells. Nonetheless, side effects of cancer therapies are often severe. They include nausea, vomiting, pain, poor appetite, wasting, cachexia, diarrhea, burning in the stomach, stress, planter warts, nerve death-neuropathy, radiation burns, fatigue, constipation, anemia, anxiety, weakened immune system, dry skin, bone marrow suppression and hair loss. As such the identification of molecules that can specifically target tumor cells, with minimal or no adverse effects to normal cells constitutes a significant area of cancer research. Such molecules with selective action against tumor cells are valuable not only for their therapeutic potential; but also for their potential applications as tools for dissection of fundamental differences between normal and cancer cells. Thus, treatment methods that specifically target certain types of hormonally linked cancers would be extremely useful. Additionally, treatment methods that target cancers located in highly vascularized tissues such as for example lung, kidney, liver, or blood, and methods that target difficult to treat cancers, such as for example melanoma, would also be highly beneficial

### OBJECT OF THE INVENTION

An object of the present invention is to provide a method of treating Cancer.

Another object of present invention is to provide a method of treating cancer in a population of cells comprising the cancer cell and normal cells.

Another object of present invention is to provide a method of treating cancer in a population of cells comprising the cancer cell and normal cells with an effective amount of agent or pharmaceutically acceptable derivative thereof for a sufficient time.

Another object of the present invention is to provide a method of treating Cancer by administering a PAR-4 inducing agent.

Another object of the present invention is to provide the use of a PAR-4 inducing agent for the treatment of Cancer.

Further object of the present invention is to provide a pharmaceutical composition comprising PAR-4 inducing agent for the treatment of Cancer.

Yet another object of the present invention is to provide the use of a PAR-4 inducing agent for inducing PAR-4 in cell.

Yet another object of the present invention is to provide the use of a PAR-4 inducing agent for inducing GRP-78 in cell.

### SUMMARY OF THE INVENTION

According to an aspect of the present invention, there is provided a method of treating cancer comprising administrating PAR-4 inducing agent.

Another aspect of present invention is to provide a method of treating cancer in a population of cells comprising the cancer cell and normal cells.

Another aspect of present invention is to provide a method of treating cancer in a population of cells comprising the cancer cell and normal cells with an effective amount of agent or pharmaceutically acceptable derivative thereof for a sufficient time.

According to another aspect of the invention, there is provided the use of a PAR-4 inducing agent for the treatment of cancer.

According to further aspect of the invention, there is provided a pharmaceutical composition comprising a PAR-4 inducing agent for the treatment of cancer.

According to an aspect of the present invention, there is provided a method of inducing PAR-4 comprising administrating PAR-4 inducing agent.

According to an aspect of the present infection, there is provided the use of a PAR-4 inducing agent for inducing GRP-78 in cell.

### BRIEF DESCRIPTION OF THE DRAWINGS

FIG. 1: Dose dependent induction of PAR-4 by Pyronaridine in Mouse embryonic fibroblast cells.

FIG. 2: Dose dependent induction of PAR-4 by Terconazole in cell supernatant of Mouse embryonic fibroblast cells.

FIG. 3: Induction of PAR-4 by Terconazole in cell lysate of Mouse embryonic fibroblast cells.

FIG. 4: Induction of PAR-4 by Mefloquine in cell supernatant of Mouse embryonic fibroblast cells.

FIG. 5: Relative to vehicle treatment, Mefloqiune induced robust elevation of PAR-4 in mouse at 51.2 mg/kg.

FIG. 6: Induction of GRP-78 by Mefloquine in ovarian and renal cancer cell lines.

FIG. 7: Induction of PAR-4 by Narasin in cell supernatant of Mouse embryonic fibroblast cells.

FIG. 8: Relative to vehicle treatment, Narasin induced robust elevation of PAR-4 in mouse at 1.5 mg/kg.

FIG. 9: Induction of PAR-4 by Mebendazole in cell supernatant and lysate of Mouse embryonic fibroblast cells.

FIG. 10: Induction of GRP-78 by Mebendazole in ovarian and renal cancer cell lines.

FIG. 11: Induction of PAR-4 by Tafenoquine in cell supernatant and lysate of Mouse embryonic fibroblast cells.

FIG. 12: Dose dependent induction of PAR-4 by Tafenoquine in cell supernatant and lysate of Mouse embryonic fibroblast cells.

FIG. 13: Induction of GRP-78 by Tafenoquine in ovarian and renal cancer cell lines.

FIG. 14: Induction of PAR-4 by Minoxidil in cell supernatant of Mouse embryonic fibroblast cells.

FIG. 15: Induction of PAR-4 by Nalidixic Acid in cell supernatant of Mouse embryonic fibroblast cells.

FIG. 16: Induction of PAR-4 by sparfloxacin in cell supernatant of Mouse embryonic fibroblast cells.

FIG. 17: Induction of PAR-4 by Pipemidic Acid in cell supernatant of Mouse embryonic fibroblast cells.

FIG. 18: Induction of PAR-4 by Lopinavir in cell supernatant of Mouse embryonic fibroblast cells.

FIG. 19: Induction of PAR-4 by Ofloxacin in cell supernatant of Mouse embryonic fibroblast cells.

### DETAILED DESCRIPTION OF THE INVENTION

The tumor suppressor PAR-4 (prostate apoptosis response-4) induces apoptotic cell death specifically in cancer cells but not in normal cells. This cancer-selective action is attributed to its centrally located SAC domain (EI-Guendy et al. (2003) “Identification of a unique core domain of PAR-4 sufficient for selective apoptosis-induction in cancer cells.” Mol. Cell. Biol. 23, 5516-5525).

The PAR-4 protein has not only an intracellular function, but it is also secreted by both normal and cancer cells (Burikhanov et al. (2009). “The tumor suppressor PAR-4 activates an extrinsic pathway for apoptosis” Cell 138, 377-388.). Secreted PAR-4 binds to its receptor GRP-78, which is upregulated on the surface of cancer cells, and induces apoptosis (Burikhanov et al. (2009); Bhattarai, T, and Rangnekar VM (2010) “Cancer-selective apoptotic effects of extracellular and intracellular PAR-4” Oncogene 29, 3873-3880). The basal level of PAR-4 secreted by normal cells is inadequate to induce apoptosis of cancer cells or inhibit the growth of tumors (Burikhanov et al. (2009)). However, elevated levels of extracellular PAR-4 produced by injecting recombinant PAR-4 in mice, cause inhibition of metastatic lung tumors (Zhao et al. (2011) “Systemic PAR-4 inhibits metastatic tumor growth”. Cancer Biol Ther. 12, 152-157).

There are several non-FDA approved small molecules, such as Nutlin-3a, PS-1145, and Arylquin-1 (INV13/1947) that can increase secretion of PAR-4 from normal cells in mice and the sera from these mice induced ex vivo apoptosis in cancer cell cultures (Burikhanov et al. (2014) “Paracrine apoptotic effect of p53 mediated by tumor suppressor PAR-4”. Cell Reports 6, 271-277; and Burikhanov et al., “Arylquin-1 targets vimentin to trigger PAR-4 secretion for tumor cell apoptosis” Nature Chem Biol. 10, 924-926 (2014)).

According to the present invention, PAR-4 inducing agents are those molecules which induce the secretion of PAR-4 from normal cells which induces apoptotic cell death specifically in cancer cells but not in normal cells.

According to the present invention, PAR-4 inducing agents are Adapalene, Narasin, Mefloquine, Mebendazole, Terconazole, Pyronaridine, Tafenoquine, Minoxidil, Nalidixic Acid, Sparfloxacin, Pipemidic Acid, Lopinavir and Ofloxacin.

Surprisingly, the inventors have found that Adapalene, Narasin, Mefloquine, Mebendazole, Terconazole, Pyronaridine, Tafenoquine, Minoxidil, Nalidixic Acid, Sparfloxacin, Pipemidic Acid, Lopinavir and Ofloxacin were found to possess PAR-4 inducing activity. In particular, described herein is the surprising effect of Adapalene, Narasin, Mefloquine, Mebendazole, Terconazole, Pyronaridine, Tafenoquine, Minoxidil, Nalidixic Acid, Sparfloxacin, Pipemidic Acid, Lopinavir and Ofloxacin including their various salt forms, to induce robust PAR-4 production from human and mouse cells and can be used to inhibit proliferation and/or metastasis of cancer cells, and to inhibit recurrent tumor formation in subjects in need thereof.

Described herein are methods for harnessing the ability of PAR-4 secreted from cells to induce apoptosis and inhibit tumor growth of cancer cells. In the methods described herein, also an aspect of this invention is a method for treating a subject having cancer cells that are p53-deficient but nonetheless unexpectedly undergo apoptosis in response to PAR-4 exposure, by administering an effective amount of PAR-4 inducing agent to the subject. An effective amount of PAR-4 inducing agent is sufficient to increase PAR-4 secretion by cells to a level that induces apoptosis of the cancer cells, and/or inhibits proliferation of such cancer cells and/or inhibits metastasis of such cancer cells. In an embodiment, the method of present invention comprises contacting a population of cells comprising cancer cells and normal cells with an effective amount of PAR-4 inducing agents or pharmaceutically acceptable derivative thereof, wherein treatment with the effective amount of PAR-4 inducing agents or pharmaceutically acceptable derivative thereof, kills cancer cells, inhibits cancer cell proliferation, cancer cell metastasis, and/or recurrence of one or more tumors.

A p53-deficient cancer cell may have a p53-deficient genotype or phenotype. For the purposes described herein a p53-deficient cancer cell has, e.g., a mutation within the p53 gene, e.g., insertion(s), deletion(s) of part or all of the gene, substitution(s) etc. such that no p53 or a mutant p53, e.g., one that does not bind DNA effectively, is produced.

Prostate apoptosis response-4 (PAR-4) is a pro-apoptotic gene identified in prostate cancer cells undergoing apoptosis. PAR-4 protein exists in the cytoplasm, endoplasmic reticulum, and nucleus. PAR-4 protein, which contains a leucine zipper domain at the carboxy-terminus, functions as a transcriptional repressor in the nucleus. In the nucleus, PAR-4 interacts with the transcription factor WT1 to inhibit the antiapoptotic protein Bcl2, and PAR-4 also inhibits the topoisomerase TOP1. In the cytoplasm, PAR-4 can be regulated by the kinases Akt and ζPKC and can inhibit the transcription factor NFκB to promote cell death. PAR-4 induces apoptosis in diverse cancer cells but not in normal cells. PAR-4 is ubiquitously expressed in normal cells and tissues. Intracellular PAR-4 is phosphorylated by protein kinase A (PKA) at a specific T155 residue found in the SAC (selective for apoptosis induction in cancer cells) domain of PAR-4. The phosphorylation of the Thr155 residue allows trafficking of the Fas/FasL to the plasma membrane. The Fas/FasL interacts with FADD causing activation of the Fas/FasL-FADD-Caspase 8 apoptotic death pathway. The basal level of PKA in normal cells is insufficient to cause T155 phosphorylation, thereby making normal cells resistant to PAR-4 mediated apoptosis.

Extracellular PAR-4 binds to its receptor GRP-78 on the cancer cell surface and induces apoptosis. In contrast, normal cells express low to undetectable levels of basal or inducible cell surface GRP-78 and are resistant to apoptosis by extracellular PAR-4. GRP-78, previously known as a prosurvival protein, is involved in PAR-4- and TRAIL induced apoptotic signaling. TRAIL binds cell surface receptors such as DR5 and DR4, which recruit FADD and caspase 8 in a DISC (death-inducing signaling complex) to initiate extrinsic cell death. Both intracellular and secreted PAR-4 play a role in apoptosis induction by caspase-dependent mechanisms.

Because the baseline levels of PAR-4 secreted by normal cells are generally inadequate to cause massive apoptosis in cancer cell cultures, secretogogues that bolster the release of PAR-4 constitute an important therapeutic advance. Nutlin-3a, originally developed as an MDM2 inhibitor, stimulated PAR-4 secretion at micromolar levels in mouse embryonic fibroblast (MEF) cells.

PAR-4 co-localized with vimentin. Vimentin is a type III intermediate filament (IF) protein that is expressed in mesenchymal cells and is the major cytoskeletal component of mesenchymal cells. Secretogogues like nutlin/arylquin 1 which cause stimulation of PAR-4 secretion exhibits this function by binding to vimentin and releasing vimentin-bound PAR-4 for secretion. This secreted PAR-4 binds to its receptor GRP-78, which is upregulated on the surface of cancer cells, and induces apoptosis. There are several non-FDA approved small molecules that can increase secretion of PAR-4 from normal cells in mice and the sera from these mice induced ex vivo apoptosis in cancer cell cultures.

According to an aspect of current invention, surprisingly the PAR-4 inducing agent was found to promote secretion of PAR-4 both in-vitro and in-vivo.

Pyronaridine is described in Croft et al Malar. J, 2012, 11, 270 which is incorporated herein by reference in its entirety.

Narasin and its biological activity is described in Miller et. al Biochem Pharmacol. 2010 May 1; 79(9): 1272-1280 which is incorporated herein by reference in its entirety.

Adapalene and its biological activity is described in Piskin & Uzunali Therapeutics and Clinical Risk Management 2007:3(4) 621-624 which is incorporated herein by reference in its entirety.

Mebendazole is described in U.S. Pat. No. 3,657,267 and Brugmans et al., J. Am. Med. Assoc. 217, 313 (1971) which are incorporated herein by reference in its entirety

Mefloquine and its preparation was first described by Ohnmacht et al. (J. Med. Chem. 1971, 14, 926) in 1971. A more detailed account of the stereochemistry, synthesis, and anti-malarial activity of the isomers of Mefloquine is given by Carroll and Blackwell (J. Med. Chem. 1974, 17, 210-219) in 1974. These references are incorporated herein by reference in its entirety.

Terconazole is described in U.S. Pat. No. 4,223,036 which is incorporated herein by reference in its entirety.

Tafenoquine is described in U.S. Pat. No. 4,617,394 which is incorporated herein by reference in its entirety.

Minoxidil is described in U.S. Pat. No. 3,382,247 which is incorporated herein by reference in its entirety.

Nalidixic acid is described in U.S. Pat. No. 3,590,036 which is incorporated herein by reference in its entirety.

Sparfloxacin is described in U.S. Pat. No. 4,795,751 which is incorporated herein by reference in its entirety.

Pipemidic acid is described in U.S. Pat. No. 3,887,557 which is incorporated herein by reference in its entirety.

Lopinavir is described in U.S. Pat. No. 5,914,332 which is incorporated herein by reference in its entirety.

Ofloxacin is described in U.S. Pat. No. 4,382,892 which is incorporated herein by reference in its entirety.

An aspect of the invention described herein is a method for treating a subject in need thereof with a PAR-4 inducing agent or pharmaceutically acceptable derivative thereof, and other agents that promote PAR-4 production by normal cells, to increase apoptosis of cancer cells and to reduce the metastasis and proliferation and/or survival of cancer cells.

By the present invention, pharmaceutically acceptable derivative thereof of PAR-4 inducing agent may include pharmaceutically acceptable derivative thereof with pharmacological activity in-vitro and/or in-vivo that is similar to the pharmacological activity of PAR-4 inducing agent as discussed herein. Such pharmaceutically acceptable derivative thereof will be readily apparent to the skilled artisan and can be readily prepared by the skilled artisan based on knowledge in the art.

The term “PAR-4 inducing agent” is used in broad sense to include not only “PAR-4 inducing agent” per se but also its pharmaceutically acceptable derivatives thereof. Suitable pharmaceutically acceptable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

The present invention includes a method of killing cancer cells, e.g., by inducing apoptosis, or inhibiting cancer cell proliferation, cancer cell metastasis, and/or recurrence of tumors in a subject comprising administering an effective amount of an agent to a subject in need of treatment for sufficient time to increase prostate apoptosis responsive 4 (PAR-4) secretion from normal cells in the subject, wherein the agent is PAR-4 inducing agent, or pharmaceutically acceptable derivative thereof, and wherein the agent may or may not administered with a chemotherapeutic.

The term “chemotherapeutic” can be used synonymously with the “anticancer” and is used in broad sense to include not only DNA-interactive Agents, Antimetabolites, Tubulin-Interactive Agents, Hormonal agents and others such as Asparaginase or hydroxyurea.

In the methods of this invention, the PAR-4 inducing agent or pharmaceutically acceptable derivative thereof comprises contacting a population of cells comprising cancer cells and normal cells with an effective amount of agent or pharmaceutically acceptable derivative thereof for a sufficient time comprises administering to a subject in need thereof in an effective amount, e.g., an amount sufficient to increase PAR-4 expression and/or secretion level above the level of PAR-4 expressed, secreted, or both by a cell prior to administration of the PAR-4 inducing agent or pharmaceutically acceptable derivative thereof by any suitable route of administration. In another aspect, an effective amount of PAR-4 inducing agent or pharmaceutically acceptable derivative thereof, is an amount sufficient to increase apoptosis of cancer cells, and/or reduce proliferation and/or reduce metastasis of cancer cells in the subject. Preferably, the PAR-4 inducing agent or salt or pharmaceutically acceptable derivative thereof is administered in an amount that induces apoptosis in cancer cells.

By way of non-limiting example, an effective amount of PAR-4 inducing agent contacted with cells may include for example about 10 nM to about 1000 μM, about 200 nM to about 100 μM, about 150 nM to about 75 μM, about 100 nM to about 50 μM, about 100 nM to about 40 μM, about 100 nM to about 35 μM, about 100 nM to about 30 μM, about 100 nM to about 25 μM.

In various embodiments, an effective amount of PAR-4 inducing agent is contacted with cells for at least about 2 hours, at least about 5 hours, at least about 10 hours, at least about 16 hours, at least about 18 hours, at least about 20 hours, at least about 24 hours, at least about 2 days, at least about 3 days, at least about 5 days, at least about a week, at least about 2 weeks, or more than 2 weeks.

By way of further non-limiting example, an effective amount of PAR-4 inducing agent administered to a subject in need thereof may include about 0.1-2000 mg daily, about 100-1500 mg daily, about 150-1200 mg daily, about 200-1200 mg daily, about 100 mg daily, about 200 mg daily, about 300 mg daily, about 400 mg daily, about 500 mg daily, about 750 mg daily, about 1000 mg daily, about 1200 mg daily or more than about 1200 mg daily.

In various embodiments, an effective amount of PAR-4 inducing agent is administered to a subject in need thereof for 1 day, for 2 days, for 5 days, for about 1 week, for about 2 weeks, for at least about 1 month, for at least about 2 months, for at least about 3 months, for at least about 6 months, for at least about 8 months, for at least about 1 year, or for more than 1 year. It is understood that the duration of treatment will depend upon the stage of cancer and whether the cancer has gone into remission.

The actual amount encompassed by the term effective amount will depend on the route of administration, the type of subject being treated, and the physical characteristics of the specific subject under consideration, e.g., their age, weight, severity of disease, comorbidities. These factors and their relationship to determining this amount are well known to skilled practitioners in the medical, and other related arts and one of skill in the art can readily determine the appropriate effective dose of PAR-4 inducing agent to be administered to a subject to achieve the desired levels of PAR-4 secretion. Likewise, the dosing schedule may be readily determined by the skilled artisan.

Preferably, PAR-4 inducing agent may be administered to the subject by any means currently used in the art, e.g., orally, subcutaneously, transdermaly, intravenously, intramuscularly, parenterally, rectally, intranasally, intratumorally etc. For example, the PAR-4 inducing agent may be administered orally to the subject in any pharmaceutically acceptable form, e.g., in the form of a tablet, a capsule, a syrup, or an elixir, or infused or injected by, e.g., an intra-peritoneal or intravenous or intramuscular route, and in an amount sufficient to increase the levels of PAR-4 in the subject, preferably to a level that is sufficient to kill the cancer cells, e.g., induce apoptosis in cancer cells, and/or inhibit proliferation and/or inhibit metastasis. Pharmaceutically acceptable forms may also comprise one or more pharmaceutically acceptable carriers, diluents, or excipients. Pharmaceutically acceptable carriers, diluents, or excipients are known in the art such as those described in, for example, Remington's Pharmaceutical Sciences, Mack Publishing Co. (A. R. Gennaro edit. 1985), incorporated herein by reference.

Preferably, PAR-4 inducing agent may be provided in the form of a pharmaceutically composition such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, spot-on, and the like), injection preparations, parenteral, topical, inhalations, buccal, nasal etc. may also be envisaged under the ambit of the invention.

The inventors of the present invention have also found that the solubility properties of the PAR-4 inducing agent are improved by nano-sizing thus leading to better bioavailability and dose reduction of the drug.

In one embodiment, the PAR-4 inducing agent may be present in the form of nanoparticles which have an average particle size of less than 2000 nm.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, surface stabilisers, channeling agents, coating agents or combinations thereof.

The cancer cells and cancers that are treated may be any type such as but not limiting to Sarcoma, Carcinoma, Leukemia, Germ Cell Tumor, Blastoma and Lymphoma and Myeloma and/or any combination thereof.

In an embodiment of the present invention, a cancer cell or tumor is located in a highly vascularized tissue. In the context of the present invention, a highly vascularized tissue includes any tissue with better than average vascularization. For example, without limitation, highly vascularized tissue includes blood, lung, liver, skin, or kidney.

A cancer cell or cancer that is located in a highly vascularized tissue may have originated in that or another highly vascularized tissue or may have travelled to a highly vascularized tissue from any other tissue by metastasis.

Preferably the PAR-4 inducing agent or pharmaceutically acceptable derivative thereof, may or may not co-administered with a chemotherapeutic or other anticancer drugs.

The methods of the present invention may also include treatment with PAR-4 inducing agent and an effective amount of ionizing radiation. Ionizing radiation has been used in the treatment of a variety of cancers, including melanoma (Khan et al. Onco Targets Ther. 2011; 4: 137-148). An effective amount of ionizing radiation may include any amount that is sufficient to kill cancer cells, inhibit cancer cell proliferation, cancer cell metastasis, and/or recurrence of one or more tumors. In an embodiment, an effective amount is about 1-5 Gy of radiation, 2-4 Gy of radiation, or about 2 Gy, about 3 Gy, about 4 Gy, or about 5 Gy of radiation. An effective amount of ionizing radiation will be apparent to the skilled artisan, and in a preferred embodiment, is less than the amount of ionizing radiation required to kill cancer cells, inhibit cancer cell proliferation, cancer cell metastasis, and/or recurrence of one or more tumors in the absence of treatment with PAR-4 inducing agent.

An effective amount of PAR-4 inducing agent may be co-administered with the ionizing radiation treatment. Improved effectiveness of the radiation therapy with administration of PAR-4 inducing agent may be manifested as an increase in cancer cell killing, e.g., cancer cell apoptosis, inhibited proliferation of cancer cells, or inhibited metastasis of cancer cells, as compared to similar subjects in need thereof treated with the same amount of ionizing radiation but without PAR-4 inducing agent treatment. The improved effectiveness of the radiation therapy with administration of PAR-4 inducing agent may alternatively or additionally be manifested by achieving cancer cell killing, e.g., cancer cell apoptosis, inhibited proliferation of cancer cells, or inhibited metastasis of cancer cells, with reduced amounts of ionizing radiation than are historically required to achieve such anti-cancer effects. In an embodiment, the effects of PAR-4 inducing agent treatment and ionizing radiation are unexpectedly more than additive.

A subject in need of treatment is a subject who has cancer, a subject who has been treated for a cancer and is in remission or cancer free, or a subject having a recurrent cancer. The cancer may be any cancer, including but not limited to, e.g., prostate, breast, skin (e.g., melanoma), and/or lung cancer, or any cancer disclosed herein. The cancer may be an androgen independent prostate cancer, a breast cancer that expresses no or low levels of PAR-4, a breast cancer that is highly aggressive, estrogen receptor-negative, high-grade (grade 3) or basal-like tumor, or a non-small cell lung cancer, a small cell lung cancer, or a lung carcinoid tumor or brain tumors. The cancer may be a melanoma, e.g., superficial spreading melanoma, nodular melanoma, lentigno maligna melanoma, or desmoplastic melanoma. A subject in need of treatment may also include a subject in need of prophylactic treatment, where a subject in need of prophylactic treatment would be, e.g., a subject who does not currently have a cancer but has been determined to be at higher risk of developing a cancer, particularly a prostate, breast, skin, or lung cancer or any cancer described herein, as compared with the risk of the general population of developing that cancer.

Also an aspect of this invention includes methods for reducing the recurrence of a cancer comprising administering PAR-4 inducing agent, or pharmaceutically acceptable derivative thereof, with or without a chemotherapeutic agent, to a subject in need thereof in an amount sufficient to reduce the recurrence of a cancer in the subject. In the methods described herein, the PAR-4 inducing agent or pharmaceutically acceptable derivative thereof may be administered to the subject in need thereof in combination with another agent that increases the production and/or secretion of PAR-4.

Another aspect of this invention is a method for prophylactically reducing the risk of a subject developing cancer comprising administering PAR-4 inducing agent, or pharmaceutically acceptable derivative thereof to a subject in need thereof to elevate PAR-4 expression from normal cells to a level that enhances apoptosis of cancer cells.

It is understood that the methods of this invention in some embodiments may include a step of selecting a subject in need thereof, by identifying a subject who has a cancer(s) described herein, or who has been treated for such cancer(s) and is in remission or cancer free, or has a recurrent cancer(s), or is in need of prophylactic treatment, and then administering an effective amount of PAR-4 inducing agent with or without an effective amount other anticancer agent, or ionizing radiation, or combinations thereof, to such subject as described herein.

### EXAMPLES

The following models and examples are for the purpose of illustration of the invention only and are not intended in any way to limit the scope of the present invention.

### Example 1. In-Vitro PAR-4 Induction by Pyronaridine

### Objective:

The objective of the current study was to check the ability of Pyronaridine to induce PAR-4 secretion from mouse embryonic fibroblast cells.

### Methodology:

Mouse embryonic fibroblast cells were harvested from exponential phase cultures. After a 24 h recovery period to allow the cells to resume exponential growth the cells were treated with M of Pyronaridine followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the Conditioned Medium (CM) as another loading control.

### Results:

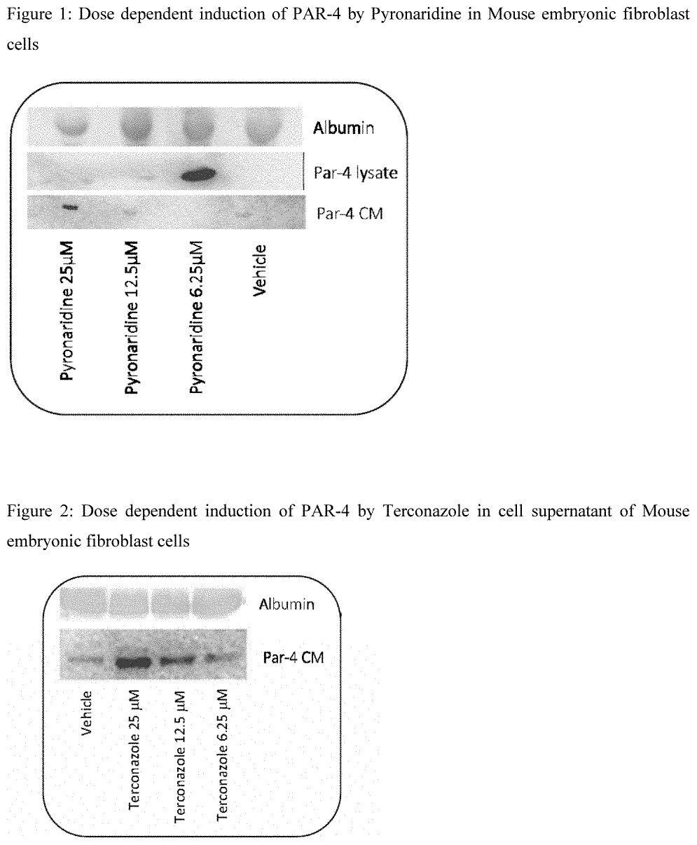

Pyronaridine was able to induce robust PAR-4 secretion in the cell supernatant from normal mouse embryonic fibroblast cells in a dose dependent manner. The results are depicted in FIG. 1.

### Example 2. In-Vitro PAR-4 Induction by Terconazole

### Objective:

The objective of the current study was to check the ability of Terconazole to induce PAR-4 secretion from mouse embryonic fibroblast cells.

### Methodology:

Mouse embryonic fibroblast cells were harvested from exponential phase cultures. After a 24 h recovery period to allow the cells to resume exponential growth the cells were treated with M of Terconazole followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the CM as another loading control.

### Results:

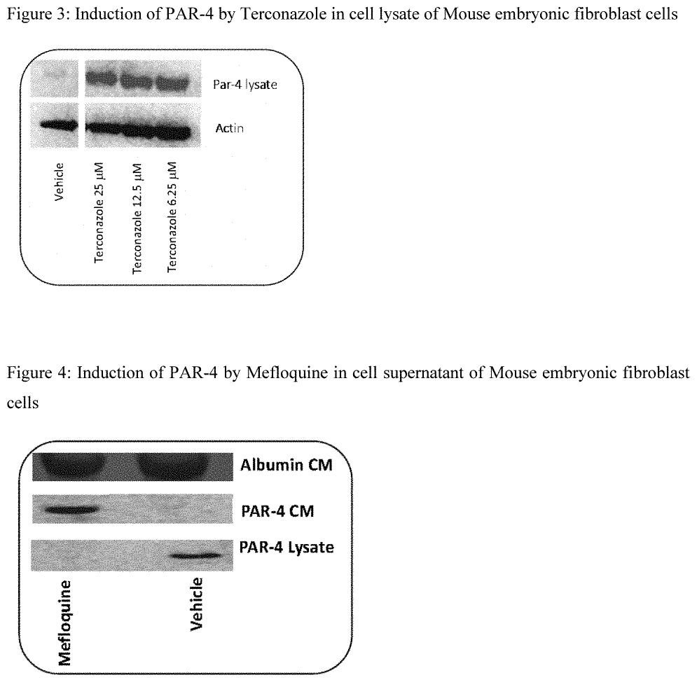

Terconazole was able to induce robust PAR-4 secretion in the cell supernatant from normal mouse embryonic fibroblast cells in a dose dependent manner. The results are depicted in FIG. 2 and FIG. 3.

### Example 3. In-Vitro PAR-4 Induction by Mefloquine

### Objective:

The objective of the current study was to check the ability of Mefloquine to induce PAR-4 secretion from mouse embryonic fibroblast cells

### Methodology:

Mouse embryonic fibroblast were harvested from exponential phase cultures. After a 24 h recovery period to allow the cells to resume exponential growth the cells were treated with 25 M of Meloquine followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the cell supernatant as another loading control. Dose dependent induction of PAR-4 by Mefloquine was also checked in Mouse embryonic fibroblast cells.

### Results:

Mefloquine was able to induce robust PAR-4 secretion in the cell supernatant from normal mouse embryonic fibroblast cells. The results are depicted in FIG. 4.

### Example 4. In-Vivo PAR-4 Induction by Mefloquine

### Objective:

The objective of the current study was to check the ability of Mefloquine to induce PAR-4 secretion systemically in C57/BL6 mice.

### Methodology:

Immunocompetent mice were orally administered with 10 mg/kg and 51.2 mg/kg body weight of Mefloquine or vehicle for 3 consecutive days followed by collection of blood 24 hrs later, serum separation and testing for systemic levels of PAR-4.

### Results:

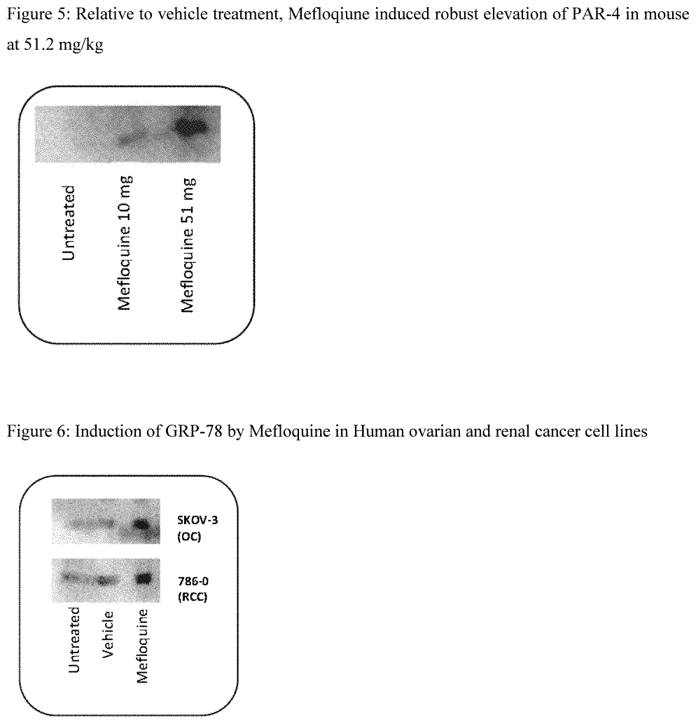

Relative to vehicle treatment, Mefloqiune induced robust elevation of PAR-4 in mouse serum and 51.2 mg/kg. The results are depicted in FIG. 5.

### Example 5. In-Vitro GRP-78 Induction by Mefloquine

### Objective:

The objective of the current study was to check the ability of Mefloquine to induce GRP-78 expression in Human ovarian cancer and renal cancer cell lines.

### Methodology:

Human ovarian cancer cell line SKOV-3 and renal cancer cell line 786-0 were harvested from exponential phase cultures. After a 24 h recovery period to allow the cells to resume exponential growth the cells were treated with 25 μM of Mefloquine followed by further 18-21 h of incubation. Induction of GRP-78 expression was checked in the cell lysate by subjecting the samples to Western blot analysis with antibodies specific for GRP-78.

### Results:

Mefloquine induced GRP-78 expression in both SKOV-3 and 786-0 at the test concentration of 25 μM. The results are depicted in FIG. 6.

### Example 6. In-Vitro PAR-4 Induction by Narasin

### Objective:

The objective of the current study was to check the ability of Narasin to induce PAR-4 secretion from mouse embryonic fibroblast cells.

### Methodology:

Mouse embryonic fibroblast cells were harvested from exponential phase cultures. After a 24 h recovery period to allow the cells to resume exponential growth the cells were treated with M of Narasin followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the CM as another loading control.

### Results:

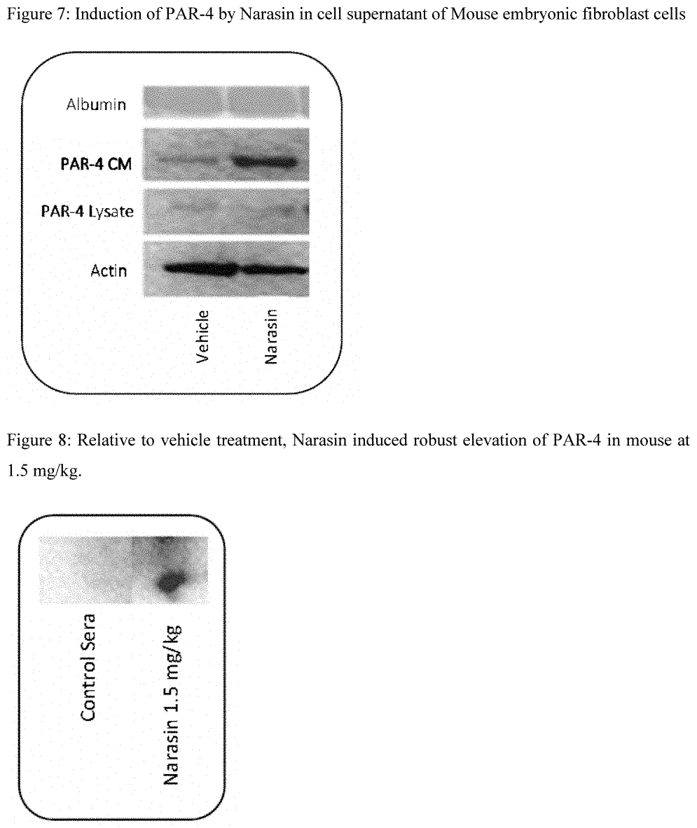

Narasin was able to induce robust PAR-4 secretion in the cell supernatant from normal mouse embryonic fibroblast cells. The results are depicted in FIG. 7.

### Example 7. In-vivo PAR-4 Induction by Narasin

### Objective:

The objective of the current study was to check the ability of Narasin to induce PAR-4 secretion systemically in C57/BL6 mice.

### Methodology:

Immunocompetent mice were orally administered with 1.5 mg/kg body weight of Narasin or vehicle for 3 consecutive days followed by collection of blood 24 hrs later, serum separation and testing for systemic levels of PAR-4. Results:

Relative to vehicle treatment, Narasin induced robust elevation of PAR-4 in mouse serum. The results are depicted in FIG. 8.

### Example 8. In-vitro PAR-4 Induction by Mebendazole

Objective:

The objective of the current study was to check the ability of Mebendazole to induce PAR-4 secretion from mouse embryonic fibroblast cells Methodology:

Mouse embryonic fibroblast were harvested from exponential phase cultures. After a 24 h recovery period to allow the cells to resume exponential growth the cells were treated with 25 μM of Mebendazole followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the cell supernatant as another loading control. Dose dependent induction of PAR-4 by Mebendazole was also checked in Mouse embryonic fibroblast cells.

### Results:

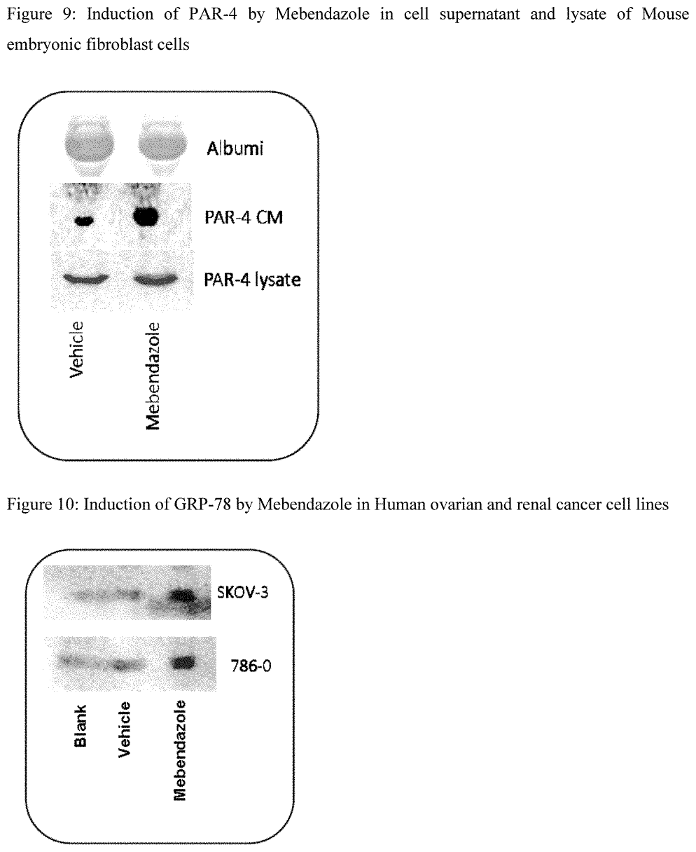

Mebendazole was able to induce robust PAR-4 secretion in the cell supernatant from normal mouse embryonic fibroblast cells. The results are depicted in FIG. 9.

### Example 9. In-Vitro GRP-78 Induction by Mebendazole

### Objective:

The objective of the current study was to check the ability of Mebendazole to induce GRP-78 expression in Human ovarian cancer and renal cancer cell lines.

### Methodology:

Human ovarian cancer cell line SKOV-3 and renal cancer cell line 786-0 were harvested from exponential phase cultures. After a 24 h recovery period to allow the cells to resume exponential growth the cells were treated with 25 μM of Mebendazole followed by further 18-21 h of incubation. Induction of GRP-78 expression was checked in the cell lysate by subjecting the samples to Western blot analysis with antibodies specific for GRP-78.

### Results:

Mebendazole induced GRP-78 expression in both SKOV-3 and 786-0 at the test concentration of 25 μM. The results are depicted in FIG. 10.

### Example 10. In-Vitro PAR-4 Induction by Tefenoquine

### Objective:

The objective of the current study was to check the ability of Tafenoquine to induce PAR-4 secretion from mouse embryonic fibroblast cells

### Methodology:

Mouse embryonic fibroblast were harvested from exponential phase cultures. After a 24 h recovery period to allow the cells to resume exponential growth the cells were treated with 25 M of Tafenoquine followed by further 18-21 h of incubation. Induction of PAR-4 was checked in the cell supernatant and cell lysate by subjecting the samples to Western blot analysis with antibodies specific for PAR-4 and actin. The samples were also subjected to SDS/PAGE and Coomassie blue staining to determine albumin levels in serum from the cell supernatant as another loading control. Dose dependent induction of PAR-4 by Tafenoquine was also checked in Mouse embryonic fibroblast cells.

### Results:

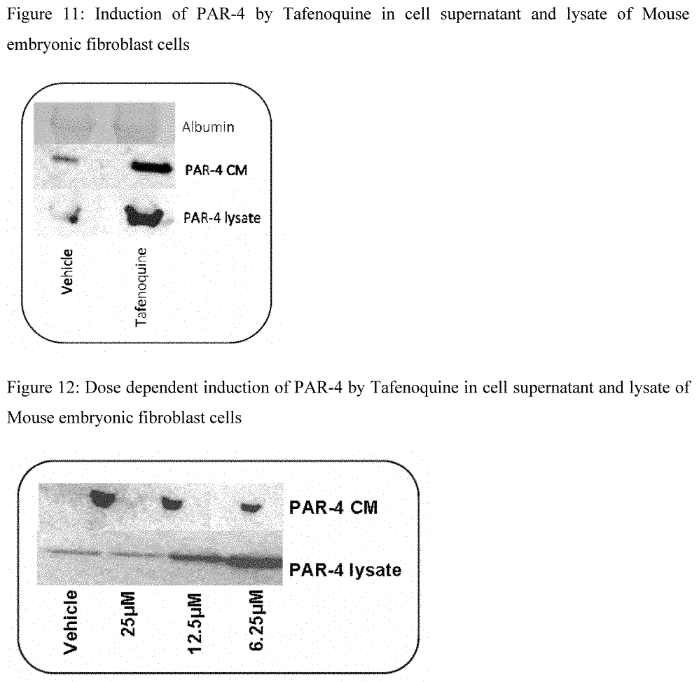

Tafenoquine was able to induce robust PAR-4 secretion in the cell supernatant from normal mouse embryonic fibroblast cells. The results are depicted in FIGS. 11 & 12

### Example 11. In-Vitro GRP-78 Induction by Tefenoquine

### Objective:

The objective of the current study was to check the ability of Tafenoquine to induce GRP-78 expression in Human ovarian cancer and renal cancer cell lines.

### Methodology:

Human ovarian cancer cell line SKOV-3 and renal cancer cell line 786-0 were harvested from exponential phase cultures. After a 24 h recovery period to allow the cells to resume exponential growth the cells were treated with 25 μM of Tafenoquine followed by further 18-21 h of incubation. Induction of GRP-78 expression was checked in the cell lysate by subjecting the samples to Western blot analysis with antibodies specific for GRP-78.

### Results:

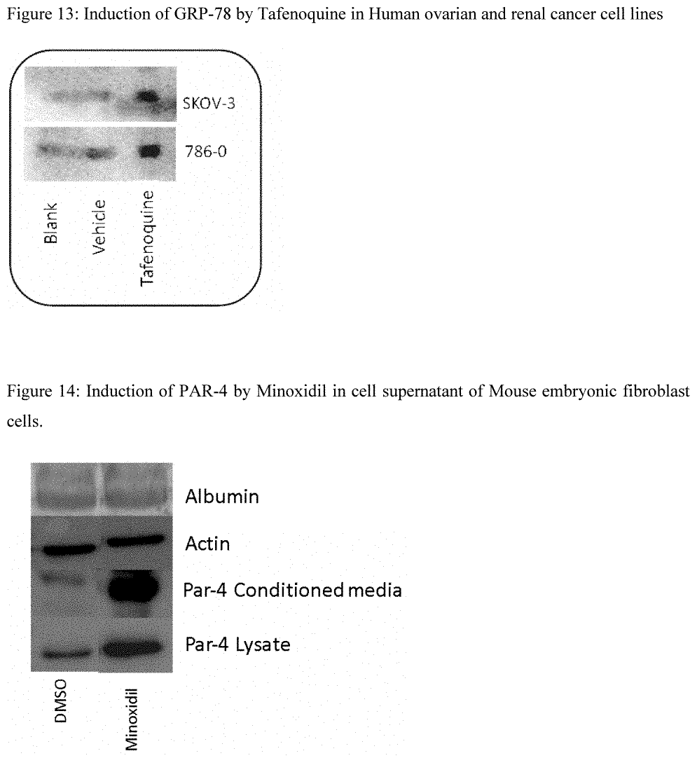

Tafenoquine induced GRP-78 expression in both SKOV-3 and 786-0 at the test concentration of 25 μM. The results are depicted in FIG. 13

### Example 12. In-Vitro PAR-4 Induction by Minoxidil

### Objective:

The objective of the current study was to check the ability of Minoxidil to induce PAR-4 secretion from mouse embryonic fibroblast cells.

### Methodology:

The experiment was conducted as per method set in an Example 1.

### Results:

Minoxidil was able to induce robust PAR-4 secretion in the cell supernatant from normal mouse embryonic fibroblast cells. The results are depicted in FIG. 14.

### Example 13. In-Vitro PAR-4 Induction by Nalidixic Acid

### Objective:

The objective of the current study was to check the ability of nalidixic acid to induce PAR-4 secretion from mouse embryonic fibroblast cells.

### Methodology:

The experiment was conducted as per method set in an Example 1.

### Results:

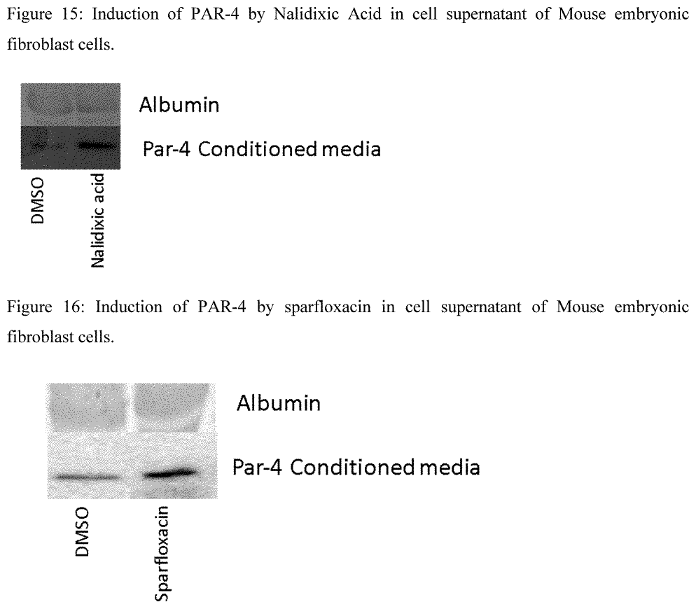

Nalidixic Acid was able to induce robust PAR-4 secretion in the cell supernatant from normal mouse embryonic fibroblast cells. The results are depicted in FIG. 15

### Example 14. In-Vitro PAR-4 Induction by Sparfloxacin

### Objective:

The objective of the current study was to check the ability of sparfloxacin to induce PAR-4 secretion from mouse embryonic fibroblast cells.

### Methodology:

The experiment was conducted as per method set in an Example 1.

### Results:

Sparfloxacin was able to induce robust PAR-4 secretion in the cell supernatant from normal mouse embryonic fibroblast cells. The results are depicted in FIG. 16.

### Example 15. In-Vitro PAR-4 Induction by Pipemidic Acid

### Objective:

The objective of the current study was to check the ability of pipemidic acid to induce PAR-4 secretion from mouse embryonic fibroblast cells.

### Methodology:

The experiment was conducted as per method set in an Example 1.

### Results:

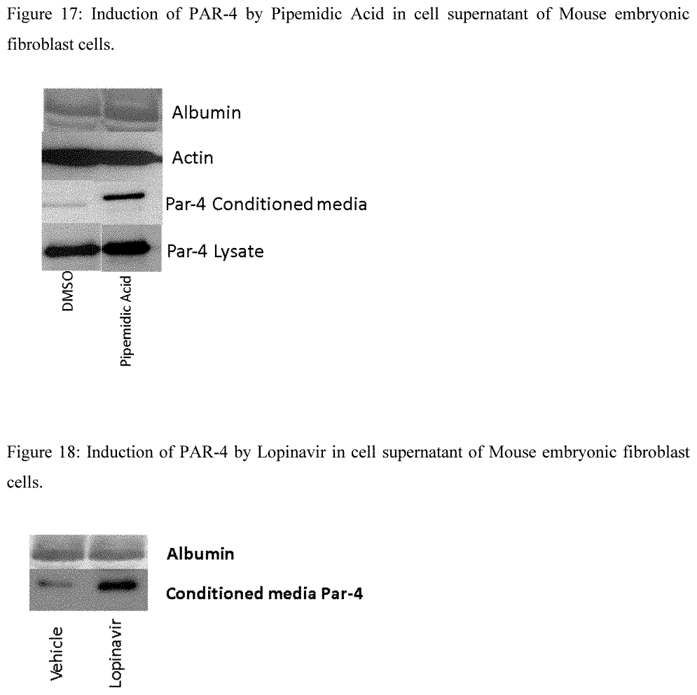

Pipemidic Acid was able to induce robust PAR-4 secretion in the cell supernatant from normal mouse embryonic fibroblast cells. The results are depicted in FIG. 17.

### Example 16. In-Vitro PAR-4 Induction by Lopinavir

### Objective:

The objective of the current study was to check the ability of Lopinavir to induce PAR-4 secretion from mouse embryonic fibroblast cells.

### Methodology:

The experiment was conducted as per method set in an Example 1.

### Results:

Lopinavir was able to induce robust PAR-4 secretion in the cell supernatant from normal mouse embryonic fibroblast cells. The results are depicted in FIG. 18.

### Example 17. In-Vitro PAR-4 Induction by Ofloxacin

### Objective:

The objective of the current study was to check the ability of ofloxacin to induce PAR-4 secretion from mouse embryonic fibroblast cells.

### Methodology:

The experiment was conducted as per method set in an Example 1.

### Results:

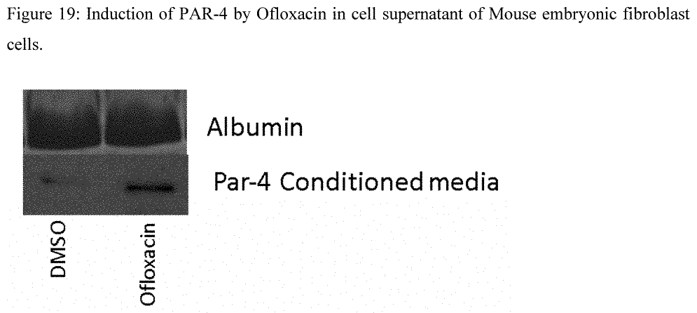

Ofloxacin was able to induce robust PAR-4 secretion in the cell supernatant from normal mouse embryonic fibroblast cells. The results are depicted in FIG. 19.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

1. A method of treating cancer, the method comprising contacting a population of cells comprising cancer cells and normal cells with an effective amount of agent or pharmaceutically acceptable derivative thereof for a sufficient time wherein such treatment induces inhibition of cancer cell proliferation, metastasis, and recurrence of one or more tumors comprising cancer cells and not in normal cells.

2. The method of claim 1, wherein the agent or pharmaceutically acceptable derivative thereof induce secretion of prostate apoptosis response-4 (PAR-4) from normal cells which induces apoptotic cell death in cancer cells but not in normal cells.

3. The method of claim 1, wherein the agent or pharmaceutically acceptable derivative thereof is selected from adapalene, narasin, mefloquine, mebendazole, terconazole, pyronaridine, tafenoquine, minoxidil, nalidixic acid, sparfloxacin, pipemidic acid, lopinavir and ofloxacin.

4. The method of claim 1, wherein the effective amount of agent or pharmaceutically acceptable derivative thereof is from about 10 nM to about 1000 μM.

5. The method of claim 4, wherein the effective amount of agent or pharmaceutically acceptable derivative thereof is from about 100 nM to about 25 μM.

6. The method of claim 1, wherein the effective amount of agent or pharmaceutically acceptable derivative thereof is from about 0.1 mg to about 2000 mg daily.

7. The method of claim 1, wherein the agent or pharmaceutically acceptable derivative thereof is contacted with the population of cells comprising cancer cells and normal cells for at least about 2 hours.

8. The method of claim 1, wherein the cancer cell is p53 deficient.

9. The method of claim 1, wherein the agent or pharmaceutically acceptable derivative thereof is in the form of tablet, capsule, syrup, elixir, infusion or injection.

10. The method of claim 1, wherein the agent or pharmaceutically acceptable derivative thereof is in the form of nanoparticles of average particle size of less than 200 nm.

11. The method of claim 1, wherein the cancer cells are sarcoma, carcinoma, leukemia, germ cell tumor, blastoma, lymphoma, myeloma cancer cell or any combination thereof.

12. The method of claim 1, wherein the cancer cell is located in a highly vascularized tissue.

13. The method of claim 1, wherein the agent or pharmaceutically acceptable derivative thereof is administered in combination with at least one other cancer therapy.

14. The method of claim 13, wherein the other cancer therapy comprises co-administration with other chemotherapeutic agent.

15. The method of claim 13, wherein the other cancer therapy comprises administering with ionizing radiation to the patient.

16. The method of claim 13, wherein the other cancer therapy comprises administering additional PAR-4 inducing agent.

17. The method of claim 1, wherein the cancer may be prostate, breast, skin, lung cancer or any combination thereof.
